(19) United States
(12) Patent Application Publication
(10) Pub. No.: US 2005/0059969 A1
(43) Pub. Date: Mar. 17, 2005

(54) Rod approximator

(75) Inventor: L. Mercer McKinley, Rancho Santa Fe, CA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: DEPUY ACROMED, INC., Raynham, MA (US)

(21) Appl. No.: 10/664,288

(22) Filed: Sep. 17, 2003

(51) Int. Cl. A61B 17/56

(52) U.S. Cl. 606/61

(57) ABSTRACT

A spinal rod approximator that is effective to approximate a spinal rod into the rod-receiving member of a spinal implant is provided. In general, the device includes first and second components that are slidably coupled to one another. The first component, hereinafter referred to as the implant-gripping member, has an implant-gripping portion that is adapted to engage the rod-receiving member of a spinal implant, and a second component, hereinafter referred to as the rod-engaging member, has rod-engaging portion that is slidably coupled to the implant-gripping member. A pusher member can be coupled to one of the implant-gripping member and the rod-engaging member, and it can be threadably mated to the other one of the implant-gripping member and the rod-engaging member. In use, the pusher member is effective to move the implant-gripping member and the rod-engaging member with respect to one another to move a spinal rod into the rod-receiving member of a spinal implant.

### FIELD OF THE INVENTION

The present invention relates to methods and devices for use in spinal surgery, and in particular to rod approximator devices and methods for using the same.

### BACKGROUND OF THE INVENTION

Spinal fixation devices are used in orthopedic surgery to align and/or fix a desired relationship between adjacent vertebral bodies. Such devices typically include a spinal fixation element, such as a relatively rigid fixation rod, that is coupled to adjacent vertebrae by attaching the element to various anchoring devices, such as hooks, bolts, wires, or screws. The fixation rods can have a predetermined contour that has been designed according to the properties of the target implantation site, and once installed, the instrument holds the vertebrae in a desired spatial relationship, either until desired healing or spinal fusion has taken place, or for some longer period of time.

Spinal fixation devices can be anchored to specific portions of the vertebra. Since each vertebra varies in shape and size, a variety of anchoring devices have been developed to facilitate engagement of a particular portion of the bone. Pedicle screw assemblies, for example, have a shape and size that is configured to engage pedicle bone. Such screws typically include a threaded shank that is adapted to be threaded into a vertebra, and a head portion having a rod-receiving element, usually in the form of a U-shaped slot formed in the head. A set-screw, plug, or similar type of fastening mechanism, is used to lock the fixation rod into the rod-receiving head of the pedicle screw. In use, the shank portion of each screw is threaded into a vertebra, and once properly positioned, a fixation rod is seated through the rod-receiving member of each screw and the rod is locked in place by tightening a cap or other fastener mechanism to securely interconnect each screw and the fixation rod.

While current spinal fixation systems have proven effective, difficulties have been encountered in introducing rods into the rod-receiving member of various fixation devices. In particular, it can be difficult to align and seat the rod into the rod receiving portion of adjacent fixation devices due to the positioning and rigidity of the spinal deformity into which the fixation device is placed and due to the desire to correct the deformity using mechanical forces applied through the rigid spinal construct. Thus, the use of a spinal rod approximator device, also sometimes referred to as a spinal rod reduction device, is often required in order to grasp the head of the fixation device and reduce or approximate the rod into the rod-receiving head of the fixation device.

While several rod approximator devices are known in the art, some tend to be difficult and very time-consuming to use. Accordingly, there is a need for improved rod approximator devices and methods for seating a spinal rod in a rod-receiving member of one or more spinal implants.

### SUMMARY OF THE INVENTION

The present invention generally provides a spinal rod approximator device for moving a spinal rod into the rod-receiving member of a spinal implant. In one embodiment, the device includes an implant-gripping member having a distal portion that extends in a direction substantially transverse to a proximal portion, and that is adapted to engage the rod-receiving member of a spinal implant. A rod-engaging member is slidably coupled to the implant-gripping member at a position proximal to the implant-gripping member, and the rod-engaging member includes a distal portion that extends transverse to a proximal portion. The device further includes a pusher member coupled to at least one of the implant-gripping member and the rod-engaging member such the pusher member is effective to move at least one the implant-gripping member and the rod-engaging member with respect to one another.

The distal portion of the rod-engaging member and the distal portion of the implant-gripping member can each have a variety of configurations. In an exemplary embodiment, the distal portion of the rod-engaging member includes opposed arms, each having a rod-receiving recess formed on a distally-facing surface thereof, and the distal portion of the implant-gripping member includes a U-shaped member having opposed legs that are adapted to be positioned under a distal end of a rod-receiving member of a spinal implant. A proximal facing surface of the U-shaped member can be substantially concave, and/or at least a portion of the U-shaped member can be substantially planar. In an exemplary embodiment, the opposed arms of the rod-engaging member are spaced apart from one another by a distance that is greater than a distance between the opposed legs of the implant-gripping member.

The pusher member can also have a variety of configurations. In one embodiment, the pusher member is fixedly, but freely-rotatably coupled to one of the implant-gripping member and the rod-engaging member, and it is threadably mated to the other one of the implant-gripping member and the rod-engaging member such that rotation of at least a portion of the pusher member is effective to move at least one of the implant-gripping member and the rod-engaging member with respect to one another. More preferably, the pusher member is a threaded rod extending through a threaded bore formed in a portion of the implant-gripping member, and wherein the threaded rod includes a distal end mated to a portion of the rod-engaging member. The threaded rod can include a handle member formed on a proximal end thereof. In another embodiment, the pusher member can be fixedly, but freely-rotatably coupled to the implant-gripping member and it can be releasably, threadably mated to the rod-engaging member. A release mechanism can be provided for releasing the threaded engagement between the pusher member and the rod-engaging member.

In yet another embodiment of the present invention, a spinal rod approximator is provided having first and second components that are slidably coupled to one another and that are adapted for relative movement along a sliding axis. The first component includes an implant-gripping portion offset from the sliding axis and adapted to engage the rod-receiving member of a spinal implant, and the second component includes a rod-engaging portion offset from the sliding axis and adapted to engage a spinal rod to move the spinal rod toward the rod-receiving member of the spinal implant being engaged by the implant-gripping portion. In an exemplary embodiment, the implant-gripping portion and the rod-engaging portion each extend in a direction substantially transverse to the sliding axis. The device can also optionally include an actuator member coupled to each of the first and second components and effective to move at least one of the components with respect to the other component. The actuator member can be, for example, an elongate rod having a threaded portion adapted to threadably couple to the first component, and having a portion rigidly mated to the second component, such that rotation of the actuator member is effective to move the second component with respect to the first component. A release mechanism can be provided for releasing the threaded engagement between the actuator member and the first component.

In other aspects, a method for approximating a spinal rod into a rod-receiving member of a spinal implant is provided using a spinal rod approximator device having an implant-gripping member and a rod-engaging member slidably coupled to one another and each having a distal portion that is offset from a sliding axis of the device. The method includes the steps of engaging a rod-receiving member of a spinal implant disposed in a patient's vertebrae with the implant-gripping member, engaging a spinal rod spaced apart from the rod-receiving member of a spinal implant with the rod-engaging member, and actuating the spinal rod approximator device to move the spinal rod engaged by the rod-engaging member into the rod-receiving member of the spinal implant engaged by the implant-gripping member.

### BRIEF DESCRIPTION OF THE DRAWINGS

The invention will be more fully understood from the following detailed description taken in conjunction with the accompanying drawings, in which:

FIG. 1 is a side view of one embodiment of a rod approximator according to the present invention;

FIG. 2 is a perspective view of the rod-engaging member of the rod approximator shown in FIG. 1;

FIG. 3 is a perspective view of the implant-gripping member of the rod approximator shown in FIG. 1;

FIG. 4 is a partial, perspective view of the back side of the rod approximator shown in FIG. 1;

FIG. 5A is a partial, perspective view of a the rod approximator device shown in FIG. 1 mated to a spinal implant and having a spinal rod extending therethrough;

FIG. 5B illustrates the system shown in FIG. 5A having the spinal rod reduced into the spinal implant by the rod approximator;

FIG. 6A illustrates a perspective view of yet another embodiment of a rod approximator device according to the present invention;

FIG. 6B is a cross-sectional view of the rod approximator shown in FIG. 6A;

FIG. 7A is a perspective view of a portion of a rod-engaging component of the rod approximator shown in FIG. 6A;

FIG. 7B is a perspective view of a portion of another rod-engaging component of the rod approximator shown in FIG. 6A;

FIG. 8 is a perspective, transparent view of a push-button mechanism of the rod approximator shown in FIG. 6A.

FIG. 9A illustrates a perspective view of another embodiment of a rod approximator in accordance with the present invention; and

FIG. 9B illustrates a perspective, transparent view of the rod approximator shown in FIG. 9A.

### DETAILED DESCRIPTION OF THE INVENTION

The present invention provides a spinal rod approximator that is effective to approximate a spinal rod into the rod-receiving member of a spinal implant. In general, the device includes first and second components that are slidably coupled to one another. The first component, hereinafter referred to as the implant-gripping member, has an implant-gripping portion that is adapted to engage the rod-receiving member of a spinal implant, and a second component, hereinafter referred to as the rod-engaging member, has rod-engaging portion that is slidably coupled to the implant-gripping member. A pusher member can be coupled to one of the implant-gripping member and the rod-engaging member, and it can be threadably mated to the other one of the implant-gripping member and the rod-engaging member. In use, the pusher member is effective to impart relative motion between the implant-gripping member and the rod-engaging member to move a spinal rod into the rod-receiving member of a spinal implant.

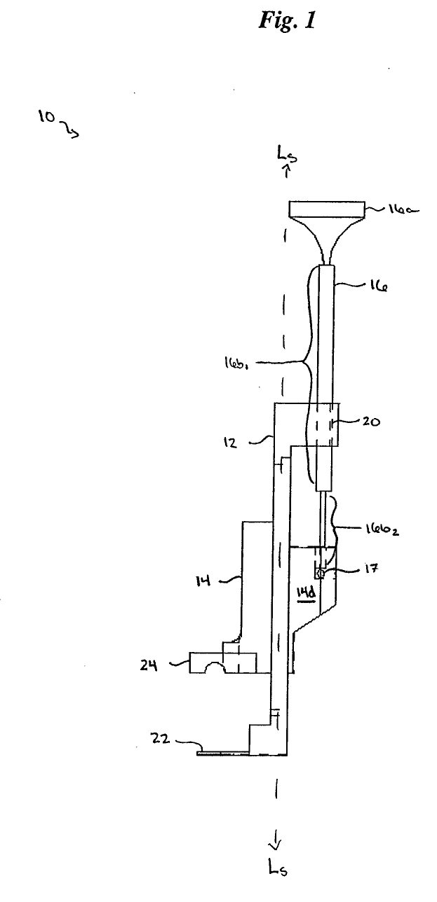

FIG. 1 illustrates one embodiment of a rod approximator device 10 in accordance with the present invention. As shown, the device 10 generally includes a rod-engaging member 14 that is mated to an implant-gripping member 12 and that is slidably movable along a sliding axis Lₛ. A pusher member 16 having external threads extends through a threaded bore 20 formed in the implant-gripping member and includes a distal end 17 that is engaged to part of the rod-engaging member 14. In use, rotation of the pusher member 16 is effective to slidably move the rod-engaging member 14 along the sliding axis Lₛ to thereby reduce a rod engaged by the rod-engaging member into the rod-receiving member of a spinal implant engaged by the implant-gripping member 12.

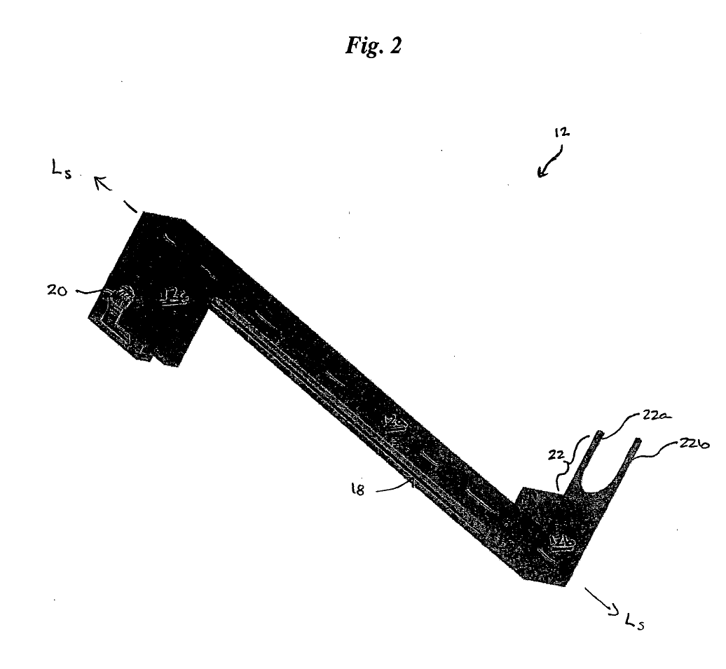

The implant-gripping member 12, which is shown in more detail in FIG. 2, can have virtually any shape and size, but it preferably includes a proximal portion 12a that is adapted to slidably mate with the rod-engaging member 14 along a longitudinal sliding axis Lₛ, and a distal portion 12b that extends in a direction substantially transverse to the sliding axis Lₛ.

The shape of the proximal portion 12a of the implant-gripping member 12 can vary, but as shown it has a generally elongate shape with a substantially rectangular or square cross-section. The proximal-most end of the proximal portion 12a can include an extension portion 12c that extends substantially transverse to the sliding axis Lₛ in a direction that is opposite to the distal portion 12b. The extension portion 12c allows the implant-gripping member 12 to mate to the pusher member 16 at a position that is offset from the sliding axis Lₛ of the implant-gripping member 12. This is particularly advantageous in that the position of the pusher member 16 does not hinder visual access to the surgical site, or access to the implant or instrument. The pusher member 16 will be discussed in more detail below.

In order to mate the proximal portion 12a of the implant-gripping member 12 to the rod-engaging member 14, the proximal portion 12a can include a longitudinal slot 18 extending therethrough for slidably receiving a portion of the rod-engaging member 14. The slot 18 preferably extends from a position adjacent the proximal-most end, e.g., from a position just distal to the extension portion 12c, and it terminates at a position that is adjacent to the distal portion 12b. This allows the rod-engaging member 14 to move a distance that is sufficient to allow a spinal rod to be engaged and moved into the rod-receiving member of a spinal implant being engaged by the implant-gripping member 12. A person skilled in the art will appreciate that a variety of other techniques can be used to slidably mate the implant-gripping member 12 and the rod-engaging member 14.

The distal portion 12b of the implant-griping member 12 can also vary, but it should be adapted to engage the rod-receiving member of a spinal implant. In an exemplary embodiment, shown in FIG. 2, the distal portion 12b of the implant-gripping member 12 includes opposed legs 22a, 22b that form a substantially U-shaped member 22. The U-shaped member 22 is preferably substantially planar to allow the opposed legs 22a, 22b to fit underneath the rod-receiving member of a spinal implant that is implanted in bone. This is particularly advantageous in that the implant-gripping member will experience smaller shear forces and it will have a more secure engagement to the rod-receiving member. In addition, gripping the implant below the rod-receiving member will serve to align a polyaxial implant to a spinal rod.

A person skilled in the art will appreciate that a variety of other techniques can be used to engage a rod-receiving member of a spinal implant, and that the engagement mechanism can vary depending on the type of implant being engaged. By way of non-limiting example, the distal portion 12b of the implant-gripping member 12 can include one or more legs that are adapted to slide into opposed slots formed on the rod-receiving member, or it can include legs that are adapted to engage a ridge formed around the rod-receiving member. In other embodiments, the distal portion 12b of the implant-gripping member 12 can include one or more pin members that fit within corresponding detents or bores formed in the rod-receiving member of a spinal implant.

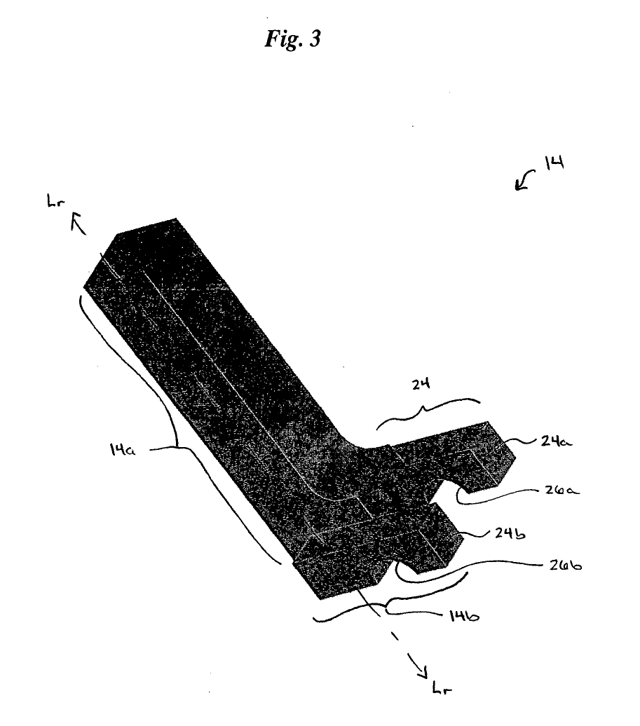

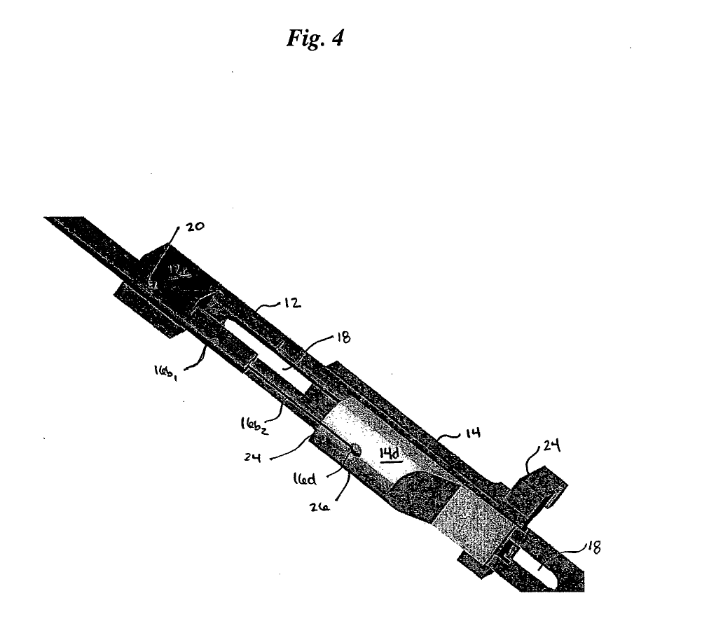

The rod-engaging member 14 of the rod approximator 10 can also have a variety of configurations, but it should be adapted to engage a spinal rod to move the rod into the rod-receiving member of a spinal implant being engaged by the implant-gripping member 12. As shown in FIG. 3, the rod-engaging member 14 includes a proximal portion 14a that extends along a longitudinal axis Lᵣ that is substantially parallel to the sliding axis Lₛ, and a distal portion 14b that extends in a direction substantially transverse to the longitudinal axis Lᵣ of the proximal portion 14a. The proximal portion 14a of the rod-engaging member 14 is adapted to slidably mate to the implant-gripping member 12. Thus, the proximal portion 14a of the rod-engaging member 14 can include a narrowed portion (not shown) that is adapted to slidably fit within the slot 18. In order to retain the connection between the proximal portion 14a of the rod-engaging member 14 and the proximal portion 12a of the implant-gripping member 12, the narrowed portion can connect to another body, e.g., bearing 14d shown in FIGS. 1 and 4, that is disposed on an opposite side of the slot 18 from the proximal portion 14a of the rod-engaging member 14. The bearing 14d not only retains the slidable connection between the rod-engaging member 14 and the implant-gripping member 12, but it also configured to mate to the pusher member 16, as will be discussed in more detail below.

The distal portion 14b of the rod-engaging member 14 is adapted to engage a spinal rod to move the rod into a rod-receiving member of a spinal implant being engaged by the implant-gripping member 12. While the distal portion 14b of the rod-engaging member 14 can have virtually any configuration, FIG. 3 illustrates opposed arms 24a, 24b that are substantially aligned with the opposed legs 22a, 22b of the implant-gripping member 12. The arms 24a, 24b, however, are preferably spaced apart from one another by a distance that is greater than a distance between the opposed legs 22a, 22b of the implant-gripping member 12. This allows a spinal rod to be moved into the rod-receiving member of a spinal implant without the arms 24a, 24b coming into contact with the spinal implant. The equal spacing between the arms 24a, 24b also provides better stability during approximation. In order to facilitate grasping of a spinal rod, each arm 24a, 24b can also include a rod-receiving recess 26a, 26b formed on a distal-facing surface thereof for seating a spinal rod. A person skilled in the art will appreciate that a variety of techniques can be used to engage a spinal rod.

As indicated above, the rod approximator device 10 also includes a pusher member 16 that is effective to move the rod-engaging member 14 and the implant-gripping member 12 with respect to one another. While a variety of techniques can be used to effect movement of the two members 12, 14, the pusher member 16 preferably includes a handle member 16a having an elongate rod 16b extending distally therefrom, as shown in FIG. 1. The rod 16b is preferably mated to one of the implant-gripping member 12 and the rod-engaging member 14, and it is movably mated to the other one of the implant-gripping member 12 and the rod-engaging member 14. As a result, movement of the pusher member 16 is effective to move one of the implant-gripping member 12 and the rod-engaging member 14.

In an exemplary embodiment, a proximal portion 16b₁ of the elongate rod 16b has external threads and extends through an internally threaded bore 20 formed in the extension member 12c of the implant-gripping member 12, and the distal portion 16b₂ of the rod 16b is fixedly, but freely-rotatably mated to the bearing 14d, which is attached to the rod-engaging member 14. As a result, rotation of the pusher member 16, e.g., using handle 16a, will controllably and mechanically move the rod-engaging member 14 along the sliding axis Lₛ with respect to the implant-gripping member 12, thereby allowing a rod to be moved into the rod-receiving member of a spinal implant being engaged by the implant-gripping member 12. While a variety of techniques can be used to mate the distal portion 16b₂ of the rod 16b of the pusher member 16 to the bearing 14d of the rod-engaging member 14, FIG. 4 illustrates an exemplary mating technique. As shown, the bearing 14d includes a channel 24 formed therein and having a distal bore 26. The channel 24 and the bore 26 are configured to freely-rotatably seat at least a portion of the distal portion 16b₂ of the pusher member 16, which includes a ball-shaped member 16d formed on the distal-most end thereof. This configuration allows the pusher member 16 to be removably mated to the rod-engaging member 14, thus allowing the device to be disassembled for cleaning. A person skilled in the art will appreciate that virtually any mating technique can be used to mate the pusher member 16 to the implant-gripping member 12 and/or to the rod-engaging member 14.

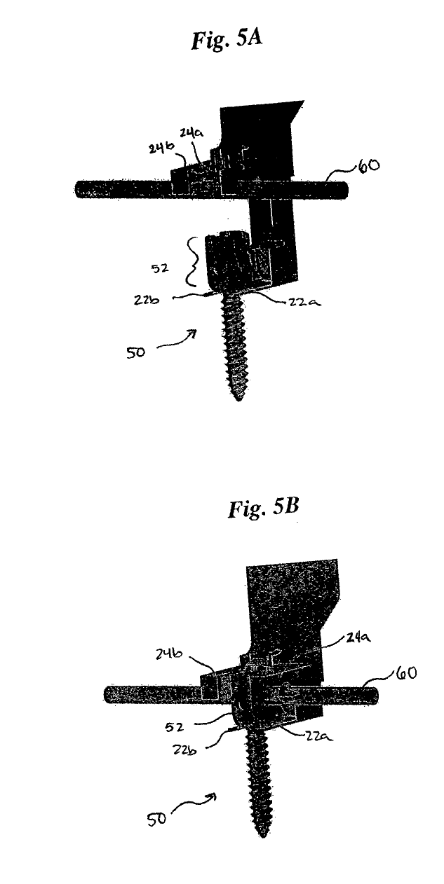

FIGS. 5A and 5B illustrate the device 10 in use. As shown in FIG. 5A, the opposed legs 22a, 22b of the implant-gripping member 12 are placed beneath the rod-receiving member 52 of a spinal implant 50, and the opposed arms 24a, 24b of the rod-engaging member 14 are placed on top of a spinal rod 60 such that the rod 60 sits within the recess 26a, 26b formed in each arm 24a, 24b. The pusher member 16 (not shown) can then be rotated to move the rod-engaging member 14 in a distal direction with respect to the implant-gripping member 12. As a result, the spinal rod 60 is pushed into the rod-receiving member 52 of the spinal implant 50 that is engaged by the opposed legs 22a, 22b of the implant-gripping member 12. A closure mechanism can then be applied to the spinal implant 50 to lock the rod 60 into the rod-receiving member 52 of the implant 50.

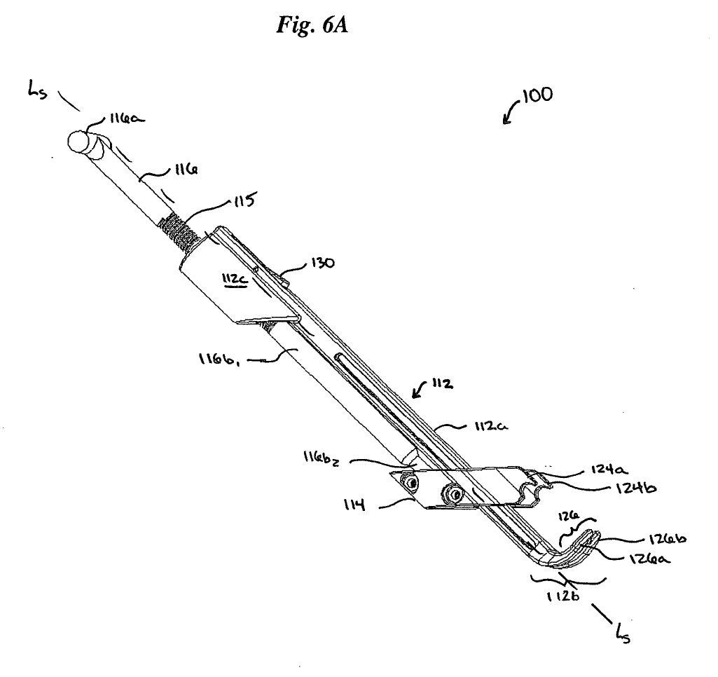

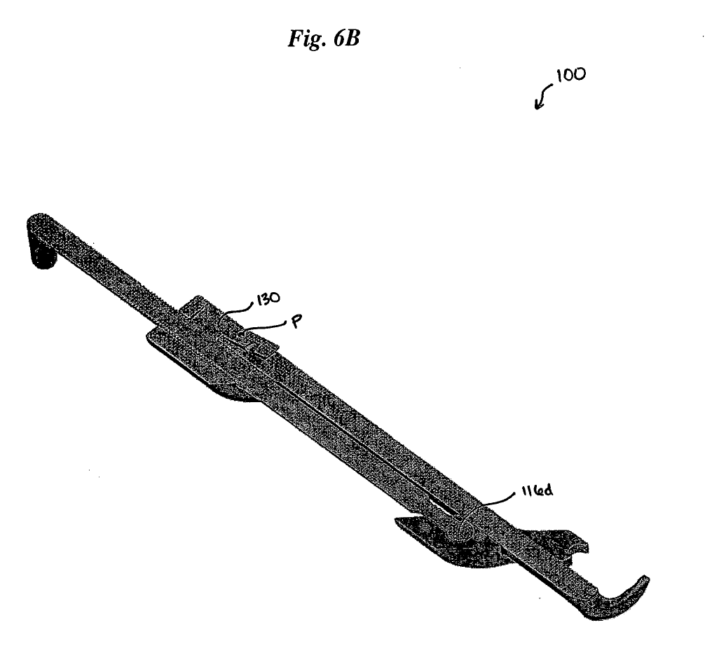

In another embodiment of the present invention, the spinal rod approximator can include a release mechanism that is effective to release the threaded engagement between the pusher member and the rod-engaging member. This allows the rod-engaging member to slid freely along the sliding axis, thereby providing the surgeon with a device that is easier to use, and more particularly it provides the surgeon with more control over the position of the rod-engaging member. By way of non-limiting example, FIGS. 6A-6B illustrate a spinal rod approximator 100 that includes an exemplary embodiment of such a release mechanism. Like reference numbers are used to refer to corresponding parts.

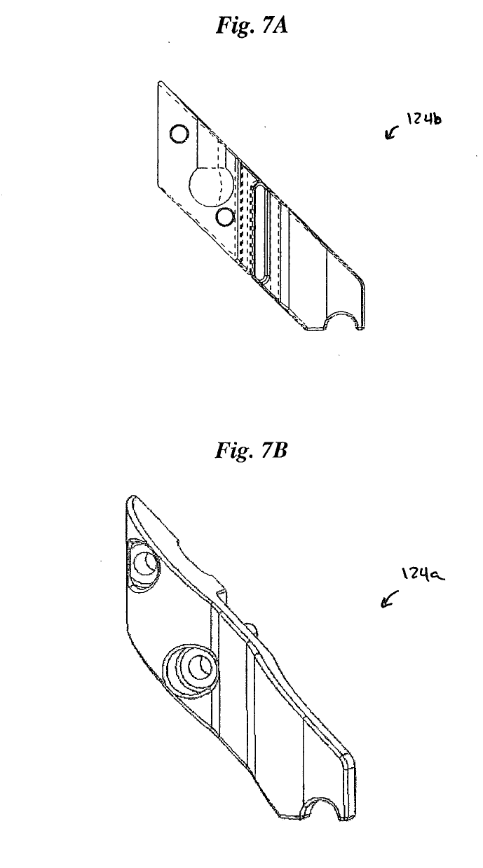

The device 100 is similar to device 10 in that it includes an implant-gripping member 112 having a proximal portion 112a and a distal portion 112b with a U-shaped implant-gripping portion 126 formed thereon, and a rod-engaging member 114 that is slidably mated to the implant-gripping member 112. The device 10 further includes a pusher member 116 having a distal end 116b₂ that fixedly, but freely-rotatably mates to the rod-engaging member 114, and a proximal portion 116b₁ that is threadably mated to an extension portion 112c of the implant-gripping member 112. One difference between devices 100 and 10 is that the rod-engaging member 114 is formed from first and second opposed rod-engaging arms 124a, 124b that extend substantially transverse to the sliding axis Lₛ of the device 100 and that mate together around the implant-gripping member 112 to slidably engage the rod-engaging member 114 to the implant-gripping member 112. The opposed arms 124a, 124b, which are shown in more detail in FIGS. 7A and 7B, also mate together to engage the distal ball-shaped member 116d therebetween. The implant-gripping member 112 also differs from implant-gripping member 12 described above in that the proximal extension portion 112c is in the form of a housing that fits around a portion of the pusher member 116. The housing 112c does not include threads formed therein, but rather it is coupled to a release mechanism 130 having threads 132 formed thereon for threadably engaging the threads 115 formed on the pusher member 116.

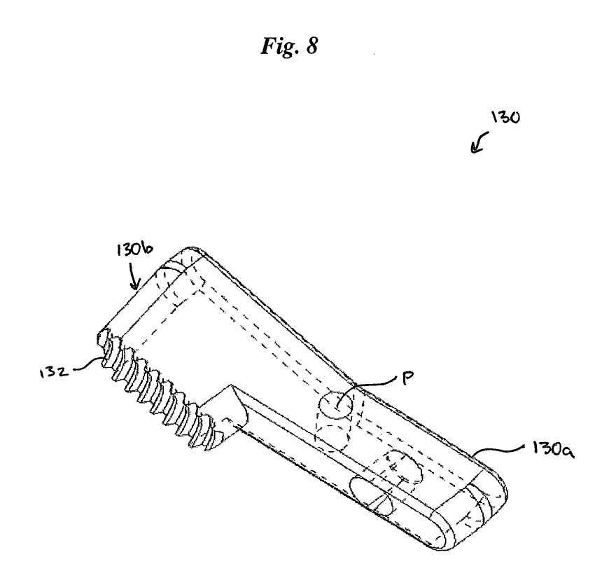

The release mechanism 130, which is shown in more detail in FIG. 8, includes a leaver-like distal end that forms a push-button 130a and a proximal end 130b having threads 132 formed on an inner surface thereof for engaging the threads 115 formed on the pusher member 116. The release mechanism 130 mates to the housing 112c such that the release mechanism is pivotable about a pivot point P, which is disposed substantially between the distal and proximal ends 130a, 130b. The release mechanism 130 further includes a biasing element (not shown), e.g., a spring, that is effective to bias the push-button 130a of the release mechanism 130 outward such that the threads 132 are forced inward to engage the threads 115 on the pusher member 116. In use, a force sufficient to overcome the biasing force can be applied to the push-button 130a to move the proximal end 130b away from the pusher member 116, thereby releasing the threaded engagement between the threads 132 on the release mechanism 130 and the threads 115 on the pusher member 116. This is particularly effective as it allows the surgeon to release the threaded engagement to freely move the rod-engaging member 114 along the sliding axis Lₛ. A person skilled in the art will appreciate that a variety of other techniques can be used to provide a mechanically releasable engagement between the pusher member 116 and the rod-engaging member 114.

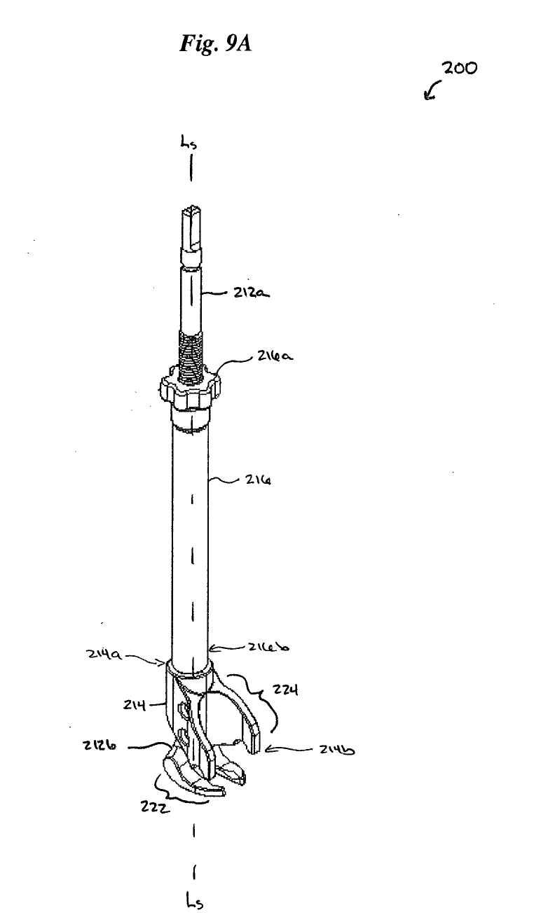

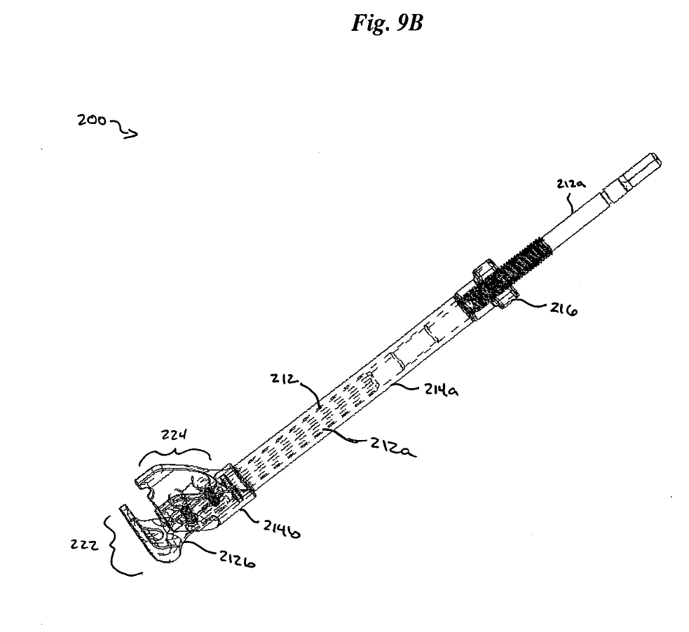

In yet another embodiment of the present invention, shown in FIGS. 9A and 9B, the spinal rod approximator 200 can have a pusher member 216 that is coaxial with, rather than offset from, the longitudinal sliding axis Lₛ of the device 200. Again, like reference numbers are used to refer to like parts. As shown, the device 200 generally includes an implant-gripping member 212 (FIG. 9B) having a proximal portion 212a and a distal portion 212b that is adapted to engage a spinal implant, and a rod-engaging member 214 having a proximal portion 214a that is disposed around the proximal portion 212a of the implant-gripping member 212, and a distal portion 214b that is adapted to engage a spinal rod. The device 200 also includes a pusher member 216 that is in the form of an elongate tube having a proximal knob or handle 216a that is disposed there around and that is threadably mated to the proximal portion 212a of the implant-gripping member 212. Rotation of the handle 216 is effective to apply a force to the proximal portion of the rod-engaging member 214, thereby moving the rod-engaging member 214 distally with respect to the implant-gripping member 212. In an exemplary embodiment, the distal end 216b of the pusher member 216 is coupled to the proximal portion 214a of the rod-engaging member 214 in a freely rotatable fashion. This allows the pusher member 216 to control movement of the rod-engaging member 214 in both a proximal and a distal direction.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

1. A spinal rod approximator device for moving a spinal rod into the rod-receiving member of a spinal implant, the device comprising:
an implant-gripping member having a distal portion that extends in a direction substantially transverse to a proximal portion, the distal portion being adapted to engage the rod-receiving member of a spinal implant;
a rod-engaging member slidably coupled to the implant-gripping member at a position proximal to the implant-gripping member, the rod-engaging member having a distal portion that extends transverse to a proximal portion; and
a pusher member coupled to at least one of the implant-gripping member and the rod-engaging member such the pusher member is effective to move at least one the implant-gripping member and the rod-engaging member with respect to one another.

2. The device of claim 1, wherein the distal portion of the rod-engaging member comprises opposed arms each having a rod-receiving recess formed on a distally-facing surface thereof.

3. The device of claim 1, wherein the distal portion of the implant-gripping member comprises a U-shaped member having opposed legs that are adapted to be positioned under a distal end of a rod-receiving member of a spinal implant.

4. The device of claim 3, wherein a proximal facing surface of the U-shaped member is substantially concave.

5. The device of claim 3, wherein at least a portion of the U-shaped member is substantially planar.

6. The device of claim 1, wherein the distal portion of the rod-engaging member comprises opposed arms, and wherein the distal portion of the implant-gripping member comprises a U-shaped member having opposed legs, the opposed arms of the rod-engaging member being spaced apart from one another by a distance that is greater than a distance between the opposed legs of the implant-gripping member.

7. The device of claim 1, wherein the pusher member is fixedly, but freely-rotatably coupled to one of the implant-gripping member and the rod-engaging member, and it is threadably mated to the other one of the implant-gripping member and the rod-engaging member such that rotation of at least a portion of the pusher member is effective to move at least one of the implant-gripping member and the rod-engaging member with respect to one another.

8. The device of claim 13, wherein the pusher member comprises a threaded rod extending through a threaded bore formed in a portion of the implant-gripping member, and wherein the threaded rod includes a distal end mated to a portion of the rod-engaging member.

9. The device of claim 8, wherein the threaded rod includes a handle member formed on a proximal end thereof.

10. The device of claim 1, wherein the pusher member is fixedly, but freely-rotatably coupled to the implant-gripping member and it is releasably, threadably mated to the rod-engaging member.

11. The device of claim 10, further comprising a release mechanism adapted to release a threaded engagement between the pusher member and the rod-engaging member.

12. A spinal rod approximator, comprising:
first and second components slidably coupled to one another and adapted for relative movement along a sliding axis, the first component including an implant-gripping portion offset from the sliding axis and being adapted to engage the rod-receiving member of a spinal implant, and the second component including a rod-engaging portion offset from the sliding axis and being adapted to engage a spinal rod to move the spinal rod toward the rod-receiving member of the spinal implant being engaged by the implant-gripping portion.

13. The spinal rod approximator of claim 12, further comprising an actuator member coupled to each of the first and second components and effective to move at least one of the components with respect to the other component.

14. The spinal rod approximator of claim 13, wherein the actuator member comprises an elongate rod having a threaded portion adapted to threadably couple to the first component, and having a portion fixedly, but freely-rotatably mated to the second component, such that rotation of the actuator member is effective to move the second component with respect to the first component.

15. The device of claim 14, further comprising a release mechanism adapted to release the threaded engagement between the actuator member and the first component.

16. The spinal rod approximator of claim 12, wherein the implant-gripping portion and the rod-engaging portion each extend in a direction substantially transverse to the sliding axis.

17. A method for approximating a spinal rod into a rod-receiving member of a spinal implant, comprising:
providing a spinal rod approximator device having an implant-gripping member and a rod-engaging member slidably coupled to one another and each having a distal portion that is offset from a sliding axis of the device;
engaging a rod-receiving member of a spinal implant disposed in a patient's vertebra with the implant-gripping member;
engaging a spinal rod spaced apart from the rod-receiving member of a spinal implant with the rod-engaging member; and
actuating the spinal rod approximator device to move the spinal rod engaged by the rod-engaging member into the rod-receiving member of the spinal implant engaged by the implant-gripping member.

18. The method of claim 17, wherein a distal portion of each of the rod-engaging member and the implant-gripping member extend in a direction substantially transverse to the sliding axis.

19. The method of claim 18, wherein the distal portion of the rod-engaging member comprises opposed arms, and wherein the distal portion of the implant-gripping member comprises a U-shaped member having opposed legs, the opposed arms of the rod-engaging member being spaced apart from one another by a distance that is greater than a distance between the opposed legs of the implant-gripping member.

20. The method of claim 18, wherein the distal portion of the rod-engaging member comprises opposed arms each having a rod-receiving recess formed on a distally-facing surface thereof.

21. The method of claim 18, wherein the distal portion of the implant-gripping member comprises a U-shaped member having opposed legs that are adapted to be positioned under a distal end of a rod-receiving member of a spinal implant.

22. The method of claim 17, wherein the device further comprises an actuator member coupled to each of the rod-engaging member and the implant-gripping member such that the actuator member is effective to move at least one of the rod-engaging member and the implant-gripping member along the sliding axis.

23. The method of claim 22, wherein the actuator member comprises a threaded rod extending through a threaded bore formed in a portion of the implant-gripping member, and wherein the threaded rod includes a distal end mated to a portion of the rod-engaging member.
